(19) United States
(12) Patent Application Publication
(10) Pub. No.: US 2008/0160548 A1
(43) Pub. Date: Jul. 3, 2008

(54) Microoptical Detection System and Method for Determination of Temperature-Dependent Parameters of Analytes

(75) Inventor: Holger Klapproth, Freiburg (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MICRONAS GMBH, Freiburg (DE)

(21) Appl. No.: 11/910,600

(22) PCT Filed: Apr. 13, 2006
(86) PCT No.: PCT/EP2006/003427

§ 371 Date: Feb. 13, 2008

(30) Foreign Application Priority Data
Apr. 20, 2005 (DE) 10 2005 018 337.9

(51) Int. Cl. G01N 33/00; G01N 21/76; C12M 1/00

(52) U.S. Cl. 435/7.92; 422/82.08; 422/52; 422/55; 435/288.7; 435/287.2; 436/172; 436/34

(57) ABSTRACT

The invention relates to a microoptical detection system and a method for detecting analytes by means of time-resolved luminescence. This serves for determination of temperature-dependent parameters of analytes, in particular for determination of point mutations of nucleic acids (DNA), for which a time-resolved detection is required.

The invention relates to a microoptical detection system and a method for detecting analytes by means of time-resolved luminescence. This serves for determination of temperature-dependent parameters of analytes, in particular for determination of point mutations of nucleic acids (DNA), for which a time-resolved detection is required.

For qualitative and/or quantitative detection of specific substances, such as e.g. biomolecules in a sample to be analysed, the use of essentially planar systems is known, which systems are described in the technical field as biosensors or biochips. These biochips form a carrier structure, on the surface of which generally a large number of detection regions which are usually disposed in a grid-like manner is configured, the individual regions or groups of regions differing from each other respectively by their specificity relative to a specific analyte to be detected. In the case of DNA analytes to be detected, there are located within the individual regions of the carrier surface—directly or indirectly immobilised—specific nucleic acid probes, such as e.g. oligonucleotides or cDNA in generally a single strand form, the respective specificity of which, relative to the nucleic acid to be detected, is prescribed essentially by the sequence order. The chip surface functionalised in this way is brought in contact, within the framework of a corresponding detection method, with the sample possibly containing the DNA analytes to be detected under conditions which, in the case of the presence of the previously detectably marked target nucleic acid(s), ensure the hybridisation thereof with the immobilised probe molecules. The qualitative and possibly quantitative detection of one or more specifically formed hybridisation complexes is effected subsequently generally by optophysical luminescence measurement and assignment of the obtained data to the respective detection fields, as a result of which determination of the presence of the DNA analyte or analytes in the sample and possibly the quantification thereof is made possible.

One field of DNA research which can only be covered insufficiently by the biochips known to date from prior art concerns the point mutations of nucleic acids. Great significance is thereby attributed in science to the identification of point mutations in the human genome. The discovery of a large number of point mutations, i.e. mutations of individual bases, which occur in more than 1% of the population, and the knowledge that point mutations can determine the side-effects of medicines leads to the vision of a person-related therapy. Accordingly, the patient is prescribed the medicine which is most compatible with him according to genotyping. A necessary condition for such a development is a rapid DNA analysis with a high throughput. However to date this has led to considerable problems in the case of the systems known from prior art since the individual base exchange occurring during a point mutation only causes a small change in the binding energy to the complementary strand. Thus the change in melting point, i.e. the temperature at which 50% of the DNA is disassociated, is generally only a few degrees Celsius, sometimes even less than 1° C.

A device for DNA analysis based on a biochip is known from DE 101 33 844 A1, receptors for forming receptor/ligand complexes being immobilised on the biochip. In addition, an excitation source and an optical detector are integrated in the device. A time-resolved analysis cannot be achieved with the system described here.

A method for parallel determination of temperature-dependent parameters, such as e.g. association/dissociation constants or equilibrium constants of complexes, is known from EP 1 248 948 B1, said method being based on total internal reflection fluorescence (TIRF). This technology which uses the evanescent field is however very complex with respect to equipment, which leads to a cost factor which is not justifiable for routine analysis. A further disadvantage which precludes systems of this type for the field of routine analysis is the high time expenditure for measurement associated with these systems.

Starting herefrom, it was the object of the present invention to provide a detection system for analytes which, on the one hand, pursues the miniaturisation known for biochips and thereby enables a time-resolved measurement at the same time. The equipment and time expenditure associated herewith of such a system is intended thereby to be kept as low as possible.

This object is achieved by the microoptical detection system having the features of claim 1, the diagnostic device having the features of claim 30 and the method for determination of temperature-dependent parameters having the features of claim 32. In claim 49, possibilities for use of the method according to the invention are mentioned. The further dependent claims reveal advantageous developments.

According to the invention, a microoptical detection system for determination of temperature-dependent parameters of analytes is provided. This is based on the following elements:
a) a carrier structure with at least one surface on which receptors for the analytes are immobilised, the receptors forming a plurality of measuring points,
b) at least one excitation source which can induce the emission of luminescent light,
c) at least one optical detector which is integrated monolithically in the carrier structure and is directed towards the surface of the carrier structure,
d) at least one device for bringing a fluid in contact with the measuring points on the surface of the carrier structure and also
e) at least one temperature-regulating element for the fluid.

The detection system according to the invention is based on the essential feature that at least the detector is integrated monolithically in the carrier structure. As a result, it is made possible that the microoptical detection system can be designed in the form of a biochip or DNA chip. Miniaturised systems of this type which make it possible, within the framework of chip technology, to develop systems which enable determination of temperature-dependent parameters are not known to date from prior art.

The detection system according to the invention can be used for the detection of nucleic acids and also for detectably marked analytes, in particular proteinaceous substances, e.g. peptides, proteins, antibodies and functional fragments of the same. Hence the present invention includes any detection of a complex formed from a detectably marked analyte, i.e. a component from the sample to be analysed, and a receptor, i.e. an immobilised carrier component, those systems also being included according to the invention in which the analyte is already characterised for example by a detectable inherent fluorescence and which therefore requires no further markings. There may be mentioned as an example in this respect the amino acid thyroxine which has inherent fluorescence and in fact even without additional marking of a proteinaceous substance which has thyroxine residues. Thus according to the invention, by using peptides as receptors, proteinaceous substances, such as e.g. antibodies or fragments of the same, can be detected as analytes, even without these requiring to be marked in advance with a suitable luminophore according to the invention.

The detection system according to the invention has a device for continually bringing in contact, said device being able to be configured preferably as a flow cell, as a cuvette or as a sample container.

With respect to the arrangement of the device for bringing the fluid in contact with the measuring points, there are basically no restrictions.

Thus a preferred variant provides that the mentioned device for bringing in contact has a channel-like configuration. It is then also possible hereby that for example a plurality of channel-like devices are integrated parallel to each other in the array-like carrier structure.

Another preferred embodiment provides that the device for bringing in contact is configured as a recess in the carrier structure, which is provided with a cover layer on the side orientated away from the surface of the carrier structure. A recess of this type can be etched for example into the carrier structure. This cover layer thereby preferably has at least two punctiform recesses which permit the inflow and outflow of the fluid.

The possibility exists in addition that the at least one device for bringing in contact comprises a photo-hardened polymer and is applied on the carrier structure by photopolymerisation.

A further preferred variant provides that a device for bringing in contact and made of any material is applied on the carrier structure, the latter being effected by means of an adhesive, by means of bonding and/or a pressing process.

Preferably the flow cell is coupled with at least one pump for the transport of fluids. Hence constant transport of the analyte or also of the washing solution along the surface of the chip takes place.

A further essential point of the detection system according to the invention is the use of a temperature-regulating element. As required, the temperature-regulating element thereby enables a temperature increase or a temperature reduction of the fluid or of the surface of the carrier structure. In order to be able to ensure this, the temperature-regulating element must be thermally connected to the fluid or at least to one surface which is in contact with the fluid. However this represents the only restriction with respect to the arrangement of the temperature-regulating element. Preferably, the temperature-regulating element is integrated monolithically in the carrier structure.

All temperature-regulating possibilities known from prior art are available for the temperature regulation of the device for bringing in contact. A preferred variant provides that the device for bringing in contact is connected to a Peltier element, as a result of which efficient heating and cooling of the device is made possible.

A further preferred embodiment of the detection system provides that the device for bringing in contact is connected to a thermosensor or temperature probe which, e.g. in combination with a controller and the temperature-regulating element, forms a control circuit. As a result, specific control of the temperature of the fluid in the device for bringing in contact is made possible.

The carrier structure of the biochip preferably comprises metal or semimetal oxides, such as e.g. silicon wafers, aluminium oxide, quartz glass, glass or a polymer.

The carrier structure of the detection system according to the invention preferably comprises a semiconductor material with an integrated optical detector layer which preferably comprises a plurality of detectors, photodiodes preferably being incorporated as detectors. In the case of a particularly preferred embodiment, the signal processing is effected at least partially within the biosensor.

The receptor can now be connected to this carrier structure both directly and via a spacer. For the connection to a spacer, i.e. a bi-functional molecule, preferably compounds are used which have a halogensilane or alkoxysilane group for coupling to the surface of the carrier structure. Amongst these, a chlorosilane group is particularly preferred. Thus for example the carrier structure can be coated with glycidyltriethoxysilane, which can be effected for example by immersion in a solution of 1% silane in toluene, slow withdrawal and immobilisation by drying at 120° C. A coating produced in this way in general has a thickness of a few Å. The coupling between spacer and receptor is effected via a suitable further functional group, for example an amino or an alkoxy group. Suitable bifunctional spacers for the coupling of a large number of different receptor molecules to a large number of carrier structure surfaces is well known to the person skilled in the art (G. T. Hermanson, “Bioconjugate Techniques”, Academic Press, 1996).

If the biomolecules to be detected concern nucleic acids, suitable DNA probes can be applied and immobilised subsequently by means of current pressure appliances.

On biosensors produced in this manner, hybridisations with e.g. biotinylated DNA can now be implemented using established methods. This can be produced for example by means of PCR and the incorporation of biotin-dUTP. During hybridisation, the biotinylated DNA now binds to the counter-strand present on the sensor (if present). Positive hybridisation occurrences can now be detected by addition of colourant conjugates, such as e.g. streptavidin/avidin conjugates. There are suitable in particular according to the invention as colourant conjugates: europium, terbium and samarium chelates, microspheres (“beads”) which are loaded for example via avidin/streptavidin with Eu-, Sm-, Tb-chelates, the mentioned chelates being characterised by their property, upon suitable excitation, of emitting luminescent light with a half-life value of the excited state of above 5 ns. Luminescent microspheres, such as e.g. FluoSpheres Europium (Molecular Probes F-20883), are hereby particularly suitable since they are able to immobilise a large number of fluorochromes with a binding occurrence. According to the invention, nanocrystals, such as are marketed for example by the Quantum Dot Corp. under the name “Quantum Dots®” are suitable in addition according to the invention.

After washing to remove non-bonded marked ligands or freely floating luminescent colourants, the measurement of the binding is effected via a suitable excitation and the measurement of the time-resolved fluorescence when the excitation light source is switched off.

Within the scope of the present invention, there are included under the term “luminescence” all light emissions, caused by an excitation source (in the further sense also the emission of ultraviolet and infrared radiation), from gaseous, liquid and solid materials which are produced not by high temperatures but by preceding energy absorption and excitation. These materials are termed luminophores. Even if the present invention is explained in more detail in part using the term “fluorescence” and “fluorophores”, these terms merely characterise preferred embodiments of the inventive basic concept and hence do not represent any restriction of the invention.

As is known to the person skilled in the art, luminescence can be caused by irradiation, from an excitation source, with light, i.e. preferably shorter wave light, and also X-rays, photoluminescence, with electrons, e.g. cathodoluminescence, ions, e.g. ionoluminescence, sound waves, e.g. sonoluminescence, with radioactive materials, e.g. radioluminescence, by electrical fields, e.g. electroluminescence, by chemical reactions, e.g. chemiluminescence or mechanical processes, e.g. triboluminescence. In contrast, thermoluminescence concerns luminescence initiated or amplified by thermal influence. All these processes are subject to the general basic laws of quantum mechanics and effect excitation of the atoms and molecules which return to the basic state subsequently with the emission of light which is detected according to the invention. The choice and possibly different design of suitable excitation sources is consequently dependent upon which type of luminescence production is intended to be applied. A preferred variant of the luminescence production within the scope of the present invention is chemiluminescence. Electronic excitation is hereby effected by a chemical reaction with subsequent light emission.

The chemiluminescence is preferably implemented in such a manner that the analytes are coupled to an enzyme marker which can catalyse the chemical reaction of a substrate with release of luminescent radiation. All enzymes which can catalyse the corresponding excitation of the substrate are suitable for this purpose, e.g. alkaline phosphatase (AP), horseradish peroxidase and other peroxidases, in particular thermostable peroxidases, glucose-6-phosphatase-dehydrogenase or xanthine oxidase. There are possible as substrates all chemiluminescent molecules, in particular luminol, isoluminol, lucigenin, peroxioxalates, acridine esters, thioesters, sulphonamides and phenanthridine esters.

A system is particularly preferred comprising horseradish peroxidase as enzyme marker, which is conjugated with a receptor for a haptene, and luminol together with hydrogen peroxide as substrate. There are possible here as receptor for example avidin or streptavidin which can then be coupled to an analyte which is biotinylated with biotin or derivatives thereof.

Another particularly preferred variant provides a system comprising alkaline phosphatase with adamantyl-1,2-dioxethanephenylphosphate as substrate. Here again, a conjugation with for example avidin, streptavidin or anti-dioxygenin as receptor is also present. These can then be coupled to analytes which are modified with the corresponding partners, such as e.g. biotin or derivates thereof and dioxygenin. It is thereby preferred that the mentioned enzymes concern temperature-stable enzymes. By using temperature-stable enzymes, it is made possible to determine the temperature-dependent parameters of the analytes.

A further variant concerns photoluminescence.

Reference is made expressly, with respect to the photoluminophores, to DE 101 33 844 A1. The organic and inorganic luminophores mentioned here can then all be used if the detection principle is based on photoluminescence.

According to one aspect of the present invention, a time-resolved fluorescence can be evaluated directly on the chip with analogue circuits in that, after switching off the excitation source, e.g. every nano-second, a value is picked up which is compared then for example also with a reference value of a previously implemented measurement which was stored likewise on the chip. In this way it is made possible in addition that non-specific interference signals, such as e.g. inherent fluorescence from possibly present system components, can be excluded. If it is assumed that resolution can take place in the interim even into the GHZ range (<1 ns) then inherent fluorescence can be distinguished from artificial fluorescence.

If the sensor surface has the design of a microarray arrangement in which a large number of detection fields are to be evaluated, the detection of the measuring field or point signal values is effected preferably sequentially in that for example entire lines or columns of the sensor surface or parts of the same are detected in succession (multiplex application).

For example the electronic output signals of the detectors can be supplied by means of suitable circuit mechanisms after an analogue-digital conversion to an external evaluation mechanism. There are possible as optical detectors or sensors which are suitable according to the invention, in addition to the photodiode (pn, p-i-n, avalanche), CCD sensors, photoconductors or a camera which are incorporated monolithically into the semiconductor substrate of the device preferably in the form of a line or array arrangement. Photodiodes can be used advantageously within the scope of a time-resolved luminescence measurement since they have a small detection surface area in comparison with photomultipliers. The use of CMOS photodiodes or CMOS cameras is hereby particularly preferred.

It is clear to the person skilled in the art that the choice of detector or of material depends upon the emission wavelength of the colourant to be detected. Basically the fact is the detector has different sensitivities with respect to the wavelength because of the so-called “semiconductor band gap” according to material choice (e.g. silicon or germanium). In the preferred case of usage of a silicon photodiode, a sensitivity region is therefore produced which extends from the infrared to the ultraviolet wave spectrum, the sensitivity being greatest between these regions (B. Streetman, Prentice-Hall, Inc., “Solid State Electronic Devices”, 1995, ISBN 0-13-436379-5, pp. 201-227).

According to a preferred embodiment, the possibly exposed surface of each photodiode comprises SiO₂ or Si₃N₄. In addition, specific method parameters of the receptor/analyte binding and of the detection can be influenced positively by the choice of surface material for the sensor chip. For example, at some points, Si₃N₄ can be applied, but at others however, SiO₂ or e.g. Al₂O₃ or a noble metal, as a result of which preferred regions on the sensor chip or even in the detection field for the biomolecules or spacers can be provided with for example more hydrophobic or more hydrophilic properties in order to promote or prevent the application of e.g. DNA receptors directed with respect to location. In addition, by applying controllable noble metal electrodes, devices preferred according to the invention can be produced, in the case of which hybridisation occurrences can be accelerated for example by applying if necessary different voltages per detection point or field or fluorescence can be initiated starting from electrically excitable colourants.

Of course the detectors can be disposed in addition in groups, as a result of which individual detection fields are produced, the input signals of which ensure as reliable a result as would be the case with individual occupation per detection region.

In addition, profiles can be picked up by a plurality of detectors per detection field, with the help of which the location-specific assignment of a binding occurrence of receptor and ligand in the course of the centring can be improved. Within the scope of these embodiments directed particularly to the microarray arrangements which are known as such, the individual photodiodes can be combined advantageously to form defined detection groups or measuring fields, as a result of which the sensitivity of the subsequent luminescence measurement and the reproducibility and reliability of the measuring data obtained consequently are significantly increased.

As a result of multiple occupation per detection field, a metrological centring of the ligand-binding occurrence can also be ensured, which can contribute in the course of the signal processing to a significant increase in sensitivity.

According to a further preferred aspect of the present invention, the excitation source is an integral component of the detection system and is provided by the detector itself. The choice of a pn diode made of direct semiconductor material makes the following possible: in the first case, the activation implies the application of a voltage, as a result of which a light signal (pn diode is used as LED) is emitted which, according to the type and nature of the pn diode, is located in a specific emission wavelength band and effects the excitation of a ligand bonded in the region of this pn diode. After deactivation of the pn diode (pn diode is used as photodiode) and after expiry of a specific waiting time, it is then activated once again in order to implement the desired measurement(s).

As a result of the fact that the excitation radiation, in the previously described embodiment, is coupled in via the same component with which the luminescence radiation is also captured, it can be achieved that a very small region of the sensor surface or of the detector field is irradiated selectively and luminescence radiation emanating from this region is evaluated. As a result of this mode of operation, the examined detector field can be imaged very precisely and interference in the measurement by the luminescence from outwith the examined region can be prevented.

The production of a sensor according to the invention can be effected by applying the CMOS (complementary metal-oxide semiconductor)—method which is known per se, as a result of which all the circuit libraries for the integration of signal conditioning and evaluation are available without modifications and can be implemented within the scope of the present invention. According to the invention, likewise suitable production methods are for example NMOS processes or bipolar processes (Wolf, Silicon Processing for the VLSI ERA, Vol. 1, Lattice Press, Sunset Beach (1986)). In addition, the possibility exists of producing a biosensor according to the invention on the basis of organic semiconductors, which is of interest in particular from the point of view of cost (EP-A-1 085 319).

According to a further preferred embodiment, the individual detection points or fields are separated from each other in such a manner that essentially no light emission of a point or field can be received by the detector or detectors of another point or field. Thus the individual detection locations can be disposed in respective depressions, as are known for example from normal microtitre plates. Trough-like depressions are preferred according to the invention and those, the lateral walls of which are disposed essentially perpendicular to the surface of the sensor chip. The respective dimensions of such a depression can be chosen freely by the person skilled in the art with knowledge of the field of application as long the luminophore or luminophores of the ligand/receptor complex to be expected are located within the depression and essentially no emission light can penetrate into adjacent depressions. A particularly preferred depression is sunk by at least 100 nm into the surface of the device according to the invention. The same effect can be achieved alternatively in that separating means which are directed perpendicularly upwards are disposed on the essentially planar detector surface, the dimensions of which separating means can be selected readily by the person skilled in the art with knowledge of the desired field of application and of the spatial dimension of an anticipated receptor/ligand complex. The application of correspondingly suitable separating means can be effected for example by anodic bonding or by so-called flip chip processes. A system of this type makes possible according to the invention a sensor-assisted electrooptical picture recording process.

In a preferred embodiment channels are applied on the detector chip so that, on one chip, a plurality of different analytes can be measured in parallel. The channels can provide for example rows of sensor elements, on which the arrays of the receptors are bonded. For example calibration measurements can be implemented thus. In a further preferred embodiment, a parallel measurement of n identical arrays is implemented in order thus to reduce the costs per analysis drastically. For this purpose, the chip is subdivided by microchannels into for example 8 identical compartments.

If a monolithically integrated semiconductor material is used as carrier mechanism for the receptors and the configuration of the sensors, a monolithically integrated circuit can also be produced on the same substrate, as a result of which preprocessing of the electronic sensor output signals can be effected in the direct vicinity of the object to be examined (receptor/analyte complex). Hence this preferred embodiment of the present invention concerns an “intelligent” sensor mechanism which achieves substantially more than purely passive sensors. For example the output signals of the electrooptical sensors can be processed by a jointly integrated circuit such that they can be guided outwards in a relatively problem-free manner via output circuits and connection contacts. In addition, the preprocessing can comprise digitalisation of the analogue sensor signals and conversion thereof into a suitable data flow. Furthermore, the signal-to-noise ratio, i.e. signal noise ratio, can be very greatly improved by the vicinity of the detector to the location of the signal processing, which is achieved in the device according to the invention, as a result of short signal paths. Furthermore, also further processing steps are possible with which for example the data quantity can be reduced or that of the external processing and display can be effected via a personal computer (PC). In addition, the device according to the invention can be configured such that the preferably compressed or processed data can be transmitted via infrared or radio connection to correspondingly equipped receiving stations.

The control of the associated mechanisms on the substrate can be effected via control signals from a control mechanism which can preferably be configured likewise entirely or partially on the substrate or is externally connected.

The possible evaluation of the optical/electrical signals within the scope of the method according to the invention via a commercially available computer has the further advantage that, via suitable programmes, an extensive automation of the data evaluation and storage is possible so that, within the scope of the data analysis, using the device according to the invention there are no restrictions relative to data generated with the help of conventional external imaging lenses.

Direct detection of the luminescences on the device according to the invention is achieved in that the receptor molecules required for a specific detection are located—directly or for example via a common spacer or coupling matrix—on the surface of an optical detector which is configured as an integral component of the device according to the invention.

The excitation source, as can be provided for example in the form of one or a plurality of white light lamps LEDs, (semiconductor) lasers, UV tubes and also by piezoelements (ultrasound) or by gases and/or liquids which emit light energy (chemical excitation), should be sufficiently powerful and preferably repeatable at high frequency. The latter property is offered when the light source can both be activated and extinguished in a short period. In the case of using an optical excitation source, this should be able to be switched off such that, after switching off, essentially no further photons impinge on the detector, such as for example by afterglow. If necessary, this can be ensured for example by using mechanical closing screens (“shutters”) and also by choosing LEDs or lasers as optical excitation source.

Preferably the excitation source with the device is coupled optically and mechanically in such a manner to the optical detector units that a radiation field is produced in the direction of the optosensors, the spatial distance of the excitation source from the detection plane being as small as possible. The distance must however be sufficient in order that the reactions between ligand and receptor which are required for the agreed use are not impaired.

On the sensor side, different or variable frequency sensors can be present for detection of the light energy emitted from a receptor/ligand complex. If photodiodes are of concern here, either wavelength-specific photoelements or else conventional photodiodes are selected, which are equipped with wavelength filters which are placed on, applied, vacuum deposited or integrated. Thus it is known for example that silicon nitride, in contrast to silicon oxide, does not allow UV light to pass through and that polysilicon absorbs UV radiation (V. P. Iordanov et al., Integrated high rejection filter for NADH fluorescence measurements, Sensors 2001 Proceedings, Vol. 1, 8-10^{th} May, pp. 106-111, AMA Service (2001)). Therefore nitride or polysilicon can be deposited on the gate oxide layer within the scope of the normal CMOS process, as a result of which corresponding filters are produced on the photodiode. Thus for example NADH (nicotine amide adenine dinucleotide) has an excitation wavelength of 350 nm and an emission wavelength of 450 nm. By applying a filter which filters out 350 nm, the sensitivity can therefore be increased. According to the invention, this effect can be used to enable differential detection in the case of parallel use of for example two different luminophores of which for example only one emits light in the UV range since the detectors provided for this purpose are configured to be UV-sensitive or not. Furthermore, this effect offers the possibility of removing from the measuring process possibly interfering inherent fluorescence of materials present with a known emission wavelength by providing corresponding filters. An example of this is the parallel use of europium chelates (emission at approx. 620 nm) and zinc sulphide doped with copper (emission at approx. 525 nm), which enable a two-colour detection as a result of emission wavelength ranges which are sufficiently different from each other, e.g. within one region of a detector point or field, in that for example half of the sensors of one detector point or field are provided with a low-pass filter and the other half of the sensors of the same point or field with a high-pass filter.

Additionally or alternatively, different luminophores can be used in parallel as long as their physical or optical properties deviate sufficiently from each other. For example, the different excitation wavelengths of two luminophores A and B to be used and/or the different half-life values thereof are used according to the invention. This can be effected for example by providing two differently doped nanocrystals.

In order to be able implement a receptor/analyte-specific detection with this layer of optosensors, it can be coated with a coupling-capable substance according to a further preferred embodiment. Typically, the sensor-chip surfaces made of metal or semimetal oxides, such as e.g. aluminium oxide, quartz glass, glass, are immersed in a solution of bifunctional molecules, so-called “linkers” which have for example a halogen silane, e.g. chlorosilane, or alkoxysilane group for coupling to the carrier structure so that a self-organising monolayer (SAM) is formed, by means of which the covalent bond between sensor surface and receptor is produced. For example coating can take place with glycidyltriethoxysilane, which can be effected for example by immersion in a solution of 1% silane in toluene, slow withdrawal and immobilisation by “baking” at 120° C. A coating produced in this way has in general a thickness of a few angstrom. The coupling between linker and receptor molecule(s) is effected via a suitable further functional group, for example an amino or epoxy group. Suitable bifunctional linkers for the coupling of a large number of different receptor molecules, in particular also of a biological origin, to a large number of carrier surfaces are well known to the person skilled in the art, cf. for example “Bioconjugate Techniques” by G. T. Hermanson, Academic Press 1996.

A diagnostic device is likewise provided according to the invention, which contains a microoptical detection system as was described previously. There should be understood by diagnostic devices hereby all the measuring arrangements for which the use of microoptical detection systems, e.g. in the form of biochips, is technically sensible and practicable. Hand-held appliances in particular are preferred here which can be used in situ, i.e. for example in the hospital or in a doctor's practice, for portable use.

According to the invention, a method for determination of temperature-dependent parameters of analytes is likewise provided. This is based on the following method steps:
A) Firstly receptors for the analytes are bonded to at least one surface of a carrier structure, the receptors forming a plurality of measuring points.
B) The receptors are brought in contact with the analytes, the result being formation of receptor-analyte complexes.
C) The receptor-analyte complexes are excited by at least one excitation source in order to effect a detectable optical change in the complex, the receptor or the analyte.
D) The optical change which is produced is then registered and subsequently evaluated with at least one detector which is integrated monolithically in the carrier structure and directed towards the surface of the carrier structure.

The previously described device is thereby used for the method according to the invention according to one of the claims 1 to 29.

A particular feature of the method according to the invention is that the excitation and the detection are effected at at least two different temperatures in order to register and evaluate the temperature-dependent parameters at the at least two temperatures.

The previously described method steps are implemented at different temperatures under otherwise identical conditions. It is therefore possible here to run a temperature programme so that the temperature is increased for example in a temperature window of 20 to 80° C. in 10° C. steps and the detection method for the analyte is implemented for these respective temperature steps. This then enables determination of a melting curve of the analytes which reproduces the temperature dependency of the dissociation of the receptor-analyte complexes.

The detection is effected preferably in the form of an ELISA, as is known from prior art.

The association constant, the dissociation constant and/or the equilibrium constant can thus be determined according to the invention as temperature-dependent parameters.

The method according to the invention is used in all fields in which determination of temperature-dependent parameters of analytes is important. Concrete examples of application fields of this type are excitation detection in the hospital, determination of paternity, criminal detection or even P450 isoenzyme analysis. In this respect, in particular malignant hyperthermia should be mentioned which is based on a mutation of the ryanodine receptor. The detection system according to the invention here can enable early detection of hyperthermia. Another important application field is the monitoring of the blood clotting cascade in order to be able to detect and treat the risk of thrombosis in good time in patients with an increased tendency towards blood clotting.

The subject according to the invention is intended to be explained in more detail by means of the subsequent Figures and the subsequent example without restricting the latter to the variants according to the invention represented here.

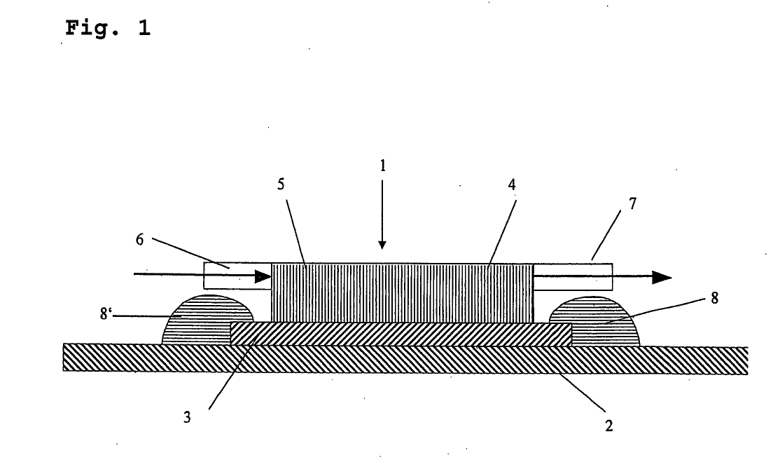

FIG. 1 shows the construction of a variant of the optical detection system according to the invention with reference to a schematic representation.

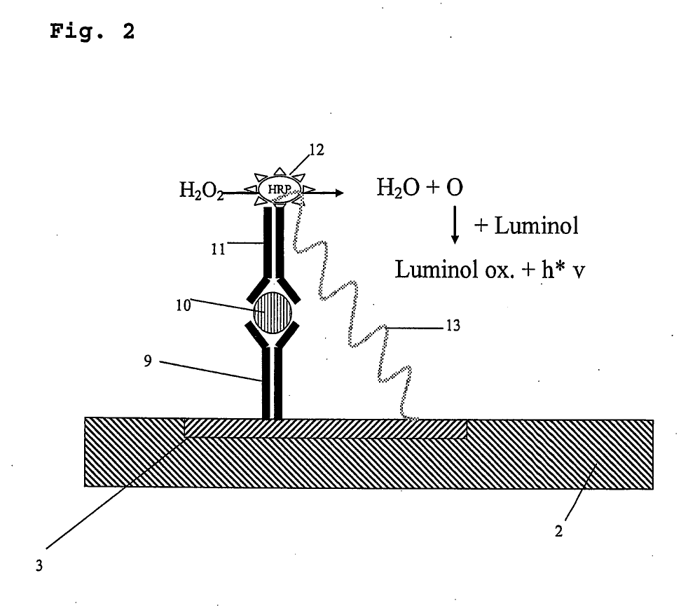

FIG. 2 shows, in a schematic representation, the detection of proteins using the microoptical detection system according to the invention.

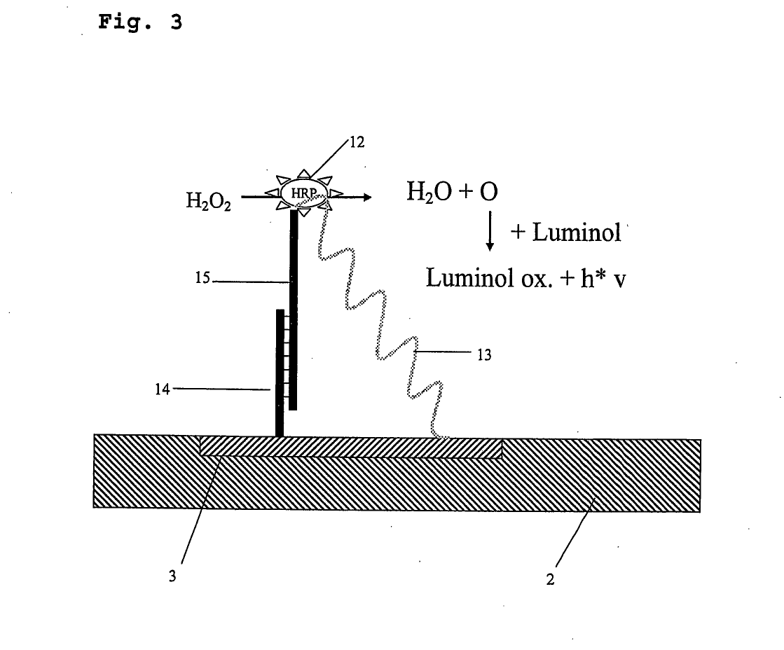

FIG. 3 shows, with reference to a schematic representation, the detection of nucleic acids with a microoptical detection system according to the invention.

A microoptical detection system 1 according to the invention is represented in FIG. 1. This is based on a carrier structure 2 which comprises the materials known from prior art for the production of chips. There are included herein in particular a carrier structure made of polychlorinated biphenylene (PCB).

A detector 3 is disposed on the carrier structure 2, in the present case in the form of a single sensor chip. This variant concerns a chemiluminescence measurement. For example, in the case of a photoluminescence measurement, in addition an excitation source can be contained in the sensor chip 3. The sensor chip 3 can be integrated also directly in the substrate. The sensor chip is mounted via two adhesion points 8 and 8′ which can comprise for example an adhesive or a solder. A flow cell 4 which serves for bringing a fluid in contact with the surface of the sensor chip continuously is disposed on the sensor chip. The flow cell thereby has an inflow 6 and an outflow 7 via which the fluid can be transported into or out of the flow cell. In addition, a temperature-regulating element 5 is integrated in the flow cell 4 and enables a temperature increase or also a temperature reduction of the fluid. Alternatively, the temperature-regulating element 5 can also be used to regulate the temperature of the surface of the carrier structure 2 or of the sensor chip. The arrangement of the temperature-regulating element 5 can be chosen arbitrarily therefore as long as the arrangement permits a corresponding temperature regulation. In the present case, a Peltier element is used as temperature-regulating element 5. In addition, the flow cell can be coupled to further components which are however not represented in the present FIG. 1. One possibility is coupling of the flow cell to a pump for transporting fluids which can be coupled directly to the inflow 6 or outflow 7.

The measuring principle for determination of proteins is represented in FIG. 2. A detector 3 in the form of a photodiode is integrated here in the carrier structure 2. A primary antibody 9 which acts as receptor is immobilised on the surface of the carrier structure 2 in the region of the photodiode 3. The receptor immobilised in this manner is then brought in contact with the sample containing the analyte 10, the result being a bond between receptor 9 and analyte 10. In a subsequent method step, the surface of the chip is then brought in contact with a detector molecule which can bond to the analyte 10. This detector molecule comprises a receptor 11, in the present case a secondary antibody, and a thermally stable enzyme 12 which is coupled hereto and can catalyse an optical detection reaction. In the present case, horseradish peroxidase is used as thermally stable enzyme. Subsequently, the substrate comprising hydrogen peroxide and luminol is then added. The light reaction associated therewith can then be registered and evaluated by means of the photodiode 3.

A carrier structure 2 in which a photodiode is integrated as detector 3 is also represented here again. A DNA receptor 14 is immobilised here in the surface of the carrier structure. In a following method step, the sample with the analytes 15 in the form of a DNA molecule is now brought in contact with the biochip. The DNA molecule can be marked for example by biotin-dUTP. In a following method step, the substrate comprising luminol and hydrogen peroxide is then added. The detection principle also corresponds here to that described under FIG. 2.

### EXAMPLE 1

Microorganisms are prepared in a suitable extraction system (Buchholz et al., 2002). In the case of the PCR, all known and also unknown bacteria can be detected with the PCR via suitable primers (consensus primer) and their 16s rRNA can be amplified. The PCR is preferably implemented asymmetrically, i.e. there is generally a lack of one of the two PCR primers in the reaction so that, in addition to double strand DNA, also single strand DNA is formed. By incorporation of biotin-dUTP in the PCR, the DNA molecules are marked. These marked molecules are then hybridised on the chip. The temperature is thereby below the melting point to be expected (e.g. 20° C. lower than the melting point). After approx. 1 h, the flow cell of the chip is rinsed with washing buffer and streptavidin-HRP is added. After approx. 10 minutes, the non-bonded HPR is removed by washing with washing buffer and ECL substrate is added (luminol plus hydrogen peroxide). After onset of the light reaction, the temperature is increased gradually from 20° C. to 80° C. and the signal is measured during each temperature step. A slow perfusion of the flow cell thereby takes place so that separated analyte no longer interferes with the measurement.

A temperature correction of the enzyme activity is implemented subsequently (the conversion of the enzyme is temperature-dependent) in order to obtain comparable measuring values. Now problematic point mutations can also be detected reliably. Since the bacteria differ greatly in the sequences between the primer, precise identification of known microorganisms can take place as a result of the choice of probes.

1. Microoptical detection system (1) for determination of temperature-dependent parameters of analytes, containing
a) a carrier structure (2) with at least one surface on which receptors (9, 14) for the analytes, for formation of receptor-analytes complexes, are immobilised, the receptors forming a plurality of measuring points,
b) at least one excitation source which can induce a detectable optical change of the receptor-analyte complex,
c) at least one optical detector (3) which is integrated monolithically in the carrier structure and is directed towards the surface of the carrier structure,
d) at least one device (4) for continuously bringing a fluid in contact with the measuring points on the surface of the carrier structure and also
e) at least one temperature-regulating element (5) for the fluid.

2. Microoptical detection system according to claim 1, characterised in that the at least one device (4) is a flow cell, a cuvette or a sample container.

3. Microoptical detection system according to one of the preceding claims,
characterised in that the at least one device (4) is etched into the carrier structure (2).

4. Microoptical detection system according to one of the preceding claims,
characterised in that the at least one device (4) comprises a photo-hardened polymer and is applied on the carrier structure (2) by photopolymerisation.

5. Microoptical detection system according to one of the preceding claims,
characterised in that the at least one device (4) is applied on the carrier structure (2) by an adhesive, bonding and/or pressing.

6. Microoptical detection system according to one of the preceding claims,
characterised in that the at least one device (4) has a channel-like configuration.

7. Microoptical detection system according to one of the preceding claims,
characterised in that the at least one device (4) is configured as a recess in the carrier structure (2) which is provided, on the side orientated away from the surface of the carrier structure (2), with a cover layer which has at least two punctiform recesses for the inflow and outflow of the fluid.

8. Microoptical detection system according to one of the preceding claims,
characterised in that the at least one device (4) is coupled to at least one pump for the transport of fluids.

9. Microoptical detection system according to one of the preceding claims,
characterised in that the temperature-regulating element (5) is a Peltier element.

10. Microoptical detection system according to one of the preceding claims,
characterised in that the temperature-regulating element (5) is thermally coupled to the device (4).

11. Microoptical detection system according to one of the preceding claims,
characterised in that the temperature-regulating element (5) is integrated monolithically in the carrier structure (2).

12. Microoptical detection system according to one of the preceding claims,
characterised in that the detection system (1) has in addition a thermosensor for determination of the temperature of the fluid and/or of the surface of the carrier structure (2).

13. Microoptical detection system according to the preceding claim,
characterised in that the thermosensor is in thermal contact with the surface of the carrier structure (2).

14. Microoptical detection system according to one of the preceding claims,
characterised in that the excitation source is a compound which is suitable for excitation of chemiluminescence.

15. Microoptical detection system according to one of the preceding claims,
characterised in that the excitation source is a radiation source for radiation, in particular light, electrons, ions and sound waves.

16. Microoptical detection system according to one of the preceding claims,
characterised in that the excitation source comprises an electrical field.

17. Microoptical detection system according to one of the preceding claims,
characterised in that the at least one detector (3) is a photodiode, a photomultiplier, a photoconductor or a camera.

18. Microoptical detection system according to one of the preceding claims,
characterised in that the at least one detector (3) is a CMOS photodiode and/or a CMOS camera.

19. Microoptical detection system according to one of the preceding claims,
characterised in that the receptors (9, 14) are bonded covalently directly or via a linker to the surface of the carrier structure (2).

20. Microoptical detection system according to one of the preceding claims,
characterised in that the linker comprises at least one layer of a bifunctional silane.

21. Microoptical detection system according to one of the preceding claims,
characterised in that the receptors (9, 14) are selected from the group comprising single and double strand nucleic acids, nucleic acid analogues, haptenes, proteins, peptides, antibodies or fragments thereof, sugar structures, receptors or ligands.

22. Microoptical detection system according to one of the preceding claims,
characterised in that the receptors (9, 14) are immobilised in detection fields which are separated from each other and represent the measuring points.

23. Microoptical detection system according to the preceding claim,
characterised in that the detection fields are disposed in an array-like manner.

24. Microoptical detection system according to one of the two preceding claims,
characterised in that the detection fields are configured as a trough-like depression in the surface of the carrier structure (2).

25. Microoptical detection system according to one of the two preceding claims,
characterised in that the detection fields are separated from each other by separating means which are aligned essentially perpendicular to the surface.

26. Microoptical detection system according to one of the preceding claims,
characterised in that at least one further component is integrated in the detection system (1), selected from the group comprising a control unit, an amplifier, a signal converter, a memory unit, a filter, a lens system, light guides and protective layers.

27. Microoptical detection system according to one of the preceding claims,
characterised in that the analyte (10, 15) is coupled to a detector molecule which contains a thermostable enzyme (12).

28. Microoptical detection system according to one of the preceding claims,
characterised in that the enzyme (12) is peroxidase or alkaline phosphatase.

29. Microoptical detection system according to one of the preceding claims,
characterised in that the analytes (10, 15) are biomolecules selected from the group comprising nucleic acids, peptides, proteins, antibodies and functional fragments thereof.

30. Diagnostic device containing a microoptical detection system according to one of the claims 1 to 27.

31. Diagnostic device according to claim 30 in the form of a hand-held device.

32. Method for determination of temperature-dependent parameters of analytes, in which
A) receptors (9, 14) for the analytes (10, 15) are bonded to at least one surface of a carrier structure (2), the receptors (9, 14) forming a plurality of measuring points,
B) the receptors (9, 14) are brought in contact with the analytes (10, 15) with formation of receptor-analyte complexes,
C) the receptor-analyte complexes are excited by at least one excitation source to effect a detectable optical change,
D) the optical change is registered and evaluated with at least one detector (3) which is integrated monolithically in the carrier structure and directed towards the surface of the carrier structure (2),
steps C) and D) being effected at at least two different temperatures in order to register and evaluate the temperature-dependent parameters at the at least two temperatures, and the method being implemented using the microoptical detection system according to one of the claims 1 to 29.

33. Method according to claim 32,
characterised in that the excitation is effected by means of light, in particular short-wave light or X-rays.

34. Method according to one of the claims 32 or 33,
characterised in that the excitation is effected by means of electrons, ions, sound waves, radioactive materials, electrical fields, induction or mechanically.

35. Method according to one of the claims 32 to 34,
characterised in that the excitation is effected by means of chemiluminescence

36. Method according to the preceding claim,
characterised in that the analyte (10, 15) is coupled to a detector molecule which comprises a thermally stable enzyme (12), which catalyses an optical detection reaction, and a receptor (11), which conjugates with the enzyme, for the analyte.

37. Method according to the preceding claim,
characterised in that the thermally stable enzyme (12) is a peroxidase and/or an alkaline phosphatase.

38. Method according to one of the claims 35 to 37,
characterised in that the temperature dependency is arithmetically corrected by means of a correction factor.

39. Method according to one of the claims 35 to 38,
characterised in that the receptor (11) is avidin and/or streptavidin and the analyte (10, 15) is biotinylated.

40. Method according to one of the claims 32 to 39,
characterised in that the detection is effected by means of photodiodes, photomultipliers, photoconductors or a camera.

41. Method according to the preceding claim,
characterised in that the detection is effected by CMOS photodiode and/or CMOS camera.

42. Method according to one of the claims 32 to 41,
characterised in that the detectors (3) are read out serially.

43. Method according to one of the claims 32 to 42,
characterised in that a thermosensor is used for determination of the temperature of the fluid and/or of the surface of the carrier structure.

44. Method according to one of the claims 32 to 43,
characterised in that the individual method steps are implemented at different temperatures under otherwise identical conditions.

45. Method according to one of the claims 32 to 44,
characterised in that, at different temperatures and before the detection, the surface of the carrier structure is rinsed with a washing solution in order to remove dissociated analytes.

46. Method according to one of the claims 32 to 45,
characterised in that the analytes (10, 15) are biomolecules selected from the group comprising nucleic acids, peptides, proteins, antibodies and functional fragments thereof.

47. Method according to one of the claims 32 to 46,
characterised in that the detection is implemented as ELISA.

48. Method-according to one of the claims 32 to 47,
characterised in that the association constant, the dissociation constant and/or the equilibrium constant are determined as temperature-dependent parameters.

49. Use of the method according to one of the claims 32 to 48, for determination of the binding strength of analytes.

50. Use according to claim 49, for determination of the association constant, the dissociation constant and/or the equilibrium constant of analytes.

51. Use according to one of the claims 49 or 50 for excitation detection in the hospital, in particular for detection of hyperthermia or monitoring of the blood clotting cascade, paternity tests, criminal detection and/or P450 isoenzyme analysis.
